(19) United States
(12) Patent Application Publication
(10) Pub. No.: US 2026/0179781 A1
(43) Pub. Date: Jun. 25, 2026

(54) ELECTROCARDIOGRAM EVALUATION METHOD

(71) Applicant: NEC Corporation, Minato-ku, Tokyo (JP)

(72) Inventors: Yuan LUO, Tokyo (JP); Mitsuru NOMA, Tokyo (JP); Osamu HISAMATSU, Tokyo (JP); Akihiko SHIBANO, Tokyo (JP); Hiroaki KATAOKA, Tokyo (JP); Kosuke NISHIHARA, Tokyo (JP); Masahiro KUBO, Tokyo (JP)

(73) Assignee: NEC Corporaiton, Minato-ku, Tokyo (JP)

(21) Appl. No.: 18/848,685

(22) PCT Filed: Mar. 29, 2022
(86) PCT No.: PCT/JP2022/015451

§ 371 Date: Sep. 19, 2024

(51) Int. Cl. G16H 50/50; A61B 5/00; A61B 5/346

(52) U.S. Cl. CPC G16H 50/50; A61B 5/346; A61B 5/4848; A61B 5/7264; A61B 5/7275

(57) ABSTRACT

A prediction apparatus of the present invention includes: a determining unit that determines an effect of treatment for a disease from electrocardiogram data before the treatment and electrocardiogram data after the treatment acquired from a person; a predicting unit that predicts a future condition of a body of the person based on a result of determining the effect of the treatment; and an output unit that outputs a result of predicting. The prediction apparatus of the present invention can support decision-making by a medical professional, for example.

### TECHNICAL FIELD

The present invention relates to an electrocardiogram evaluation method, an electrocardiogram evaluation apparatus, and a program.

### BACKGROUND ART

One method for diagnosing a physical condition is to use an electrocardiogram. For example, in medical institutions, diagnosis of a physical condition is performed by measuring a 12-lead electrocardiogram or a monitored electrocardiogram with an electrocardiogramd evaluating the waveform of the electrocardiogram. Then, in recent years, as described in Patent Literature 1, an electrocardiogram is automatically analyzed and evaluated using a model generated through machine learning. For example, in Patent Literature 1, evaluation of an electrocardiogram is performed by generating a model by learning normal electrocardiograms and anomalous electrocardiograms of various diseases such as myocardial infarction, and inputting a measured electrocardiogram into the model.

### CITATION LIST

### Patent Literature

Patent literature 1: Japanese Unexamined Patent Application Publication No. JP-A 2020-130772

### SUMMARY OF INVENTION

However, it is possible to diagnose a physical condition at the time using an electrocardiogram, but it is difficult to accurately predict a future physical condition of the same person. In particular, in a case where a person has a disease, prognosis prediction is important, but it is difficult to make an accurate prediction.

An object of the present invention is to provide a prediction method which can solve the abovementioned problem that it is difficult to accurately predict a future physical condition using an electrocardiogram.

### Solution to Problem

A prediction method as an aspect of the present invention includes: determining an effect of treatment for a disease from electrocardiogram data before the treatment and electrocardiogram data after the treatment acquired from a person; predicting a future condition of a body of the person based on a result of determining the effect of the treatment; and outputting a result of predicting.

Further, a prediction apparatus as an aspect of the present invention includes: a determining unit that determines an effect of treatment for a disease from electrocardiogram data before the treatment and electrocardiogram data after the treatment acquired from a person; a predicting unit that predicts a future condition of a body of the person based on a result of determining the effect of the treatment; and an output unit that outputs a result of predicting.

Further, a computer program as an aspect of the present invention includes instructions for causing a computer to execute processes to:
determine an effect of treatment for a disease from electrocardiogram data before the treatment and electrocardiogram data after the treatment acquired from a person;
predict a future condition of a body of the person based on a result of determining the effect of the treatment; and
output a result of predicting.

### Advantageous Effects of Invention

Configured as described above, the present invention enables accurate prediction of a future physical condition using an electrocardiogram.

### BRIEF DESCRIPTION OF DRAWINGS

FIG. 1 is a view showing the overall configuration of an information processing system in a first example embodiment of the present invention.

FIG. 2 is a block diagram showing the configuration of a prediction apparatus disclosed in FIG. 1.

FIG. 3 is a flowchart showing the operation of the prediction apparatus disclosed in FIG. 1.

FIG. 4 is a block diagram showing the hardware configuration of a prediction apparatus in a second example embodiment of the present invention.

FIG. 5 is a block diagram showing the configuration of the prediction apparatus in the second example embodiment of the present invention.

FIG. 6 is a flowchart showing the operation of the prediction apparatus in the second example embodiment of the present invention.

### DESCRIPTION OF EXAMPLE EMBODIMENTS

### First Example Embodiment

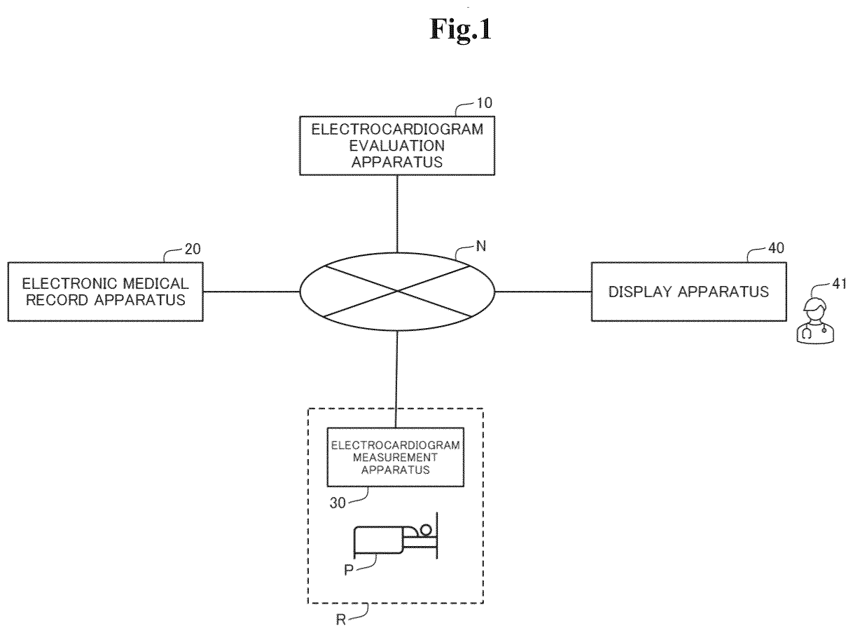

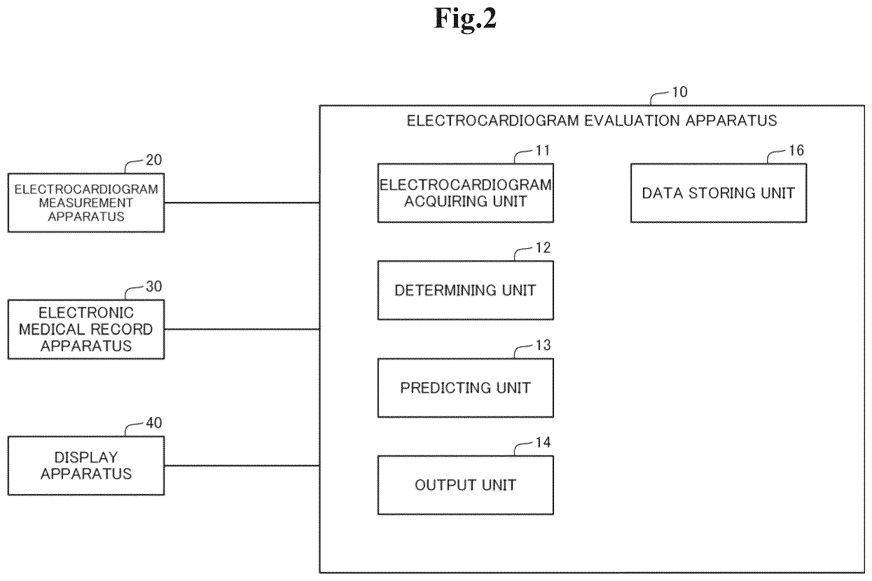

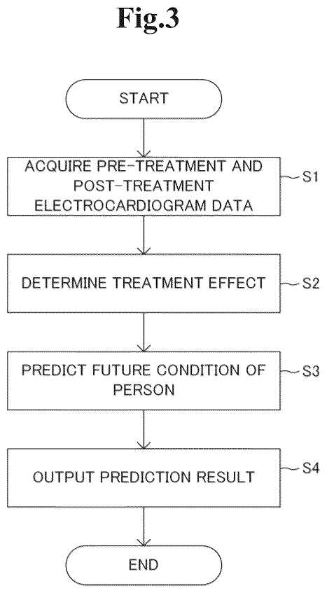

A first example embodiment of the present invention will be described with reference to FIGS. 1 to 3. FIGS. 1 and 2 are views for describing the configuration of an information processing system, and FIG. 3 is a view for describing the processing operation of the information processing system.

### Configuration

The information processing system of the present invention is for evaluating an electrocardiogram in order to diagnose a physical condition of a person in a medical institution. For example, the information processing system determines an effect of treatment from electrocardiograms before and after treatment, and predicts a future condition from the result of determining the effect of the treatment.

As shown in FIG. 1, the information processing system includes an electrocardiogram evaluation apparatus 10, an electronic medical record apparatus 20, an electrocardiogram measurement apparatus 30, and a display apparatus 40, which are connected via a network N. The respective components will be described in detail below.

The electrocardiogram measurement apparatus 30 is an apparatus that measures an electrocardiogram from a person P. For example, the electrocardiogram measurement apparatus 30 is an electrocardiogramnstalled in a predetermined location R in a medical institution, such as a hospital room, an examination room and an intensive care unit, or a monitor electrocardiogram be worn by the person P, or a wearable device such as a wristwatch-type mobile terminal. In this example embodiment, the electrocardiogram measurement apparatus 30 is installed in a medical institution and is capable of measuring a 12-lead electrocardiogram.

In addition to the configuration to measure an electrocardiogram, the electrocardiogram measurement apparatus 30 also includes a configuration included by a general information processing apparatus, such as a communication device and an arithmetic logic unit, and also includes a function to transmit measured electrocardiogram data to the electronic medical record apparatus 20. Consequently, electrocardiogram data measured by the electrocardiogram measurement apparatus 30 is stored into an electronic medical record of each person P. As an example, an operator of the electrocardiogram measurement apparatus 30 specifies an electronic medical record of a person P who is a measurement target from among electronic medical records stored in the electronic medical record apparatus 20, and records the electrocardiogram data of the person P into the electronic medical record. In addition, in a case where the electrocardiogram measurement apparatus 30 is a wearable device, the electrocardiogram measurement apparatus 30 transmits electrocardiogram data to the electronic medical record apparatus 20 together with identification information of a person P, and records the electrocardiogram data into an electronic medical record corresponding to the person P. However, electrocardiogram data may be recorded into an electronic medical record by any method.

Further, when recording electrocardiogram data into the electronic medical record apparatus 20, the electrocardiogram measurement apparatus 30 transmits identification information such as its own IP address to the electronic medical record apparatus 20 in association with the electrocardiogram data. That is to say, the electrocardiogram measurement apparatus 30 transmits electrocardiogram data in association with identification information indicating the sender of the electrocardiogram data. At this time, the identification information may be data that identifies a location where the electrocardiogram measurement apparatus 30 that is the sender of the electrocardiogram data is installed, that is, a location of measurement of the electrocardiogram. For example, by previously assigning different identification information to the respective electrocardiogram measurement apparatuses 30 installed in locations R such as a hospital room, an examination room and an intensive care unit, and storing correspondence information between the respective locations R and the respective identification information in the electronic medical record apparatus 20 or the electrocardiogram evaluation apparatus 10 to be described later, it is possible to identify a location of measurement of electrocardiogram data from identification information associated with the electrocardiogram data. Also, in a case where the electrocardiogram measurement apparatus 30 is a wearable device, associating identification information such as an IP address or information indicating a wearable device with electrocardiogram data makes it possible to identify that the electrocardiogram data has been measured by the wearable device.

In addition, electrocardiogram data measured by the electrocardiogram measurement apparatus 30 may be directly transmitted to the electrocardiogram evaluation apparatus 10.

The electronic medical record apparatus 20 is configured with a general information processing apparatus including an arithmetic logic unit and a memory unit managed by a medical institution, and stores an electronic medical record of a person P in the memory unit. For example, the examination result and diagnosis result of the person P are recorded in the electronic medical record. As an example, in the electronic medical record, the following data of the person P are recorded: basic physical data such as age, gender, height, and weight; measurement data such as heart rate, body temperature, blood pressure, and the electrocardiogram data mentioned above; examination data such as blood test result and image diagnosis result; and medical condition data such as state of consciousness, current or past disease, condition at the time of diagnosis, and condition at the time of examination. Data in the electronic medical record is recorded by input of data by a diagnostician or an examiner, or by transmission of data from the electrocardiogram measurement apparatus 30 such as an electrocardiogram a wearable device mentioned above or from various examination devices and measurement devices.

At this time, the electrocardiogram data to be recorded in the electronic medical record is recorded in association with time information such as the date and time when the electrocardiogram has been measured. Moreover, disease information representing the name of a disease and symptoms of the disease that the person P is suffering from at that time is associated with the electrocardiogram data and recorded. The time information and the disease information associated with the electrocardiogram data may be information recorded in the electronic medical record.

The display apparatus 40 is a general information processing apparatus that is managed by a medical institution and includes an arithmetic logic unit and a memory unit operated by a medical professional 41 such as a doctor. When the medical professional 41 diagnoses a person P, the display apparatus 40 instructs the electrocardiogram evaluation apparatus 10 to predict the course of treatment in response to an operation by the medical professional 41. For example, when accepting input of information specifying electrocardiogram data before treatment and electrocardiogram data after treatment of a target person P from the medical professional 41, the display apparatus 40 transmits the information specifying the electrocardiogram data and also an instruction to predict the course of treatment of the person P to the electrocardiogram evaluation apparatus 10. Then, as will be described later, the display apparatus 40 outputs the course of treatment of the target person P predicted by the electrocardiogram evaluation apparatus 10, and presents it to the medical professional 41.

The electrocardiogram evaluation apparatus 10 (prediction apparatus) is configured with one or a plurality of information processing apparatuses each including an arithmetic logic unit and a memory unit. Then, as shown in FIG. 2, the electrocardiogram evaluation apparatus 10 includes an electrocardiogram acquiring unit 11, a determining unit 12, a predicting unit 13, and an output unit 14. The respective functions of the electrocardiogram acquiring unit 11, the determining unit 12, the predicting unit 13, and the output unit 14 can be realized by the arithmetic logic unit executing a program for realizing the respective functions stored in the memory unit. In addition, the electrocardiogram evaluation apparatus 10 includes a data storing unit 16. The data storing unit 16 is configured with the memory unit. The respective components will be described in detail below.

At the time of diagnosis of a person P, the electrocardiogram acquiring unit 11 acquires electrocardiogram data of a corresponding person P from the electronic medical record apparatus 20 described above and stores it into the data storing unit 16. At this time, the electrocardiogram acquiring unit 11 accepts, along with an instruction to predict the course of treatment, an instruction to specify electrocardiogram data before treatment and electrocardiogram data after treatment of the person P from the medical professional 41 via the display apparatus 40, and acquires the electrocardiogram data from the electronic medical record apparatus 20. The electrocardiogram acquiring unit 11 accepts, for example, an instruction to specify a date and time before treatment and a date and time after treatment, and acquires electrocardiogram data at the corresponding dates and times. The electrocardiogram data before treatment and the electrocardiogram data after treatment may each be electrocardiogram data at a specific date and time or electrocardiogram data at a plurality of dates and times.

In addition, the electrocardiogram acquiring unit 11 may also acquire any record data recorded in the electronic medical record of the person P and store it into the data storing unit 16. For example, the electrocardiogram acquiring unit 11 may acquire basic physical data such as age, gender, height and weight, measurement data such as heart rate, body temperature and blood pressure, and medical condition data such as state of consciousness, current or past disease, a condition at the time of diagnosis and a condition at the time of examination, which are recorded in the electronic medical record of the person P.

The determining unit 12 determines an effect of treatment from electrocardiogram data before treatment and electrocardiogram data after treatment of the person P. For example, the determining unit 12 compares the waveform of the electrocardiogram data before treatment with the waveform of the electrocardiogram data after treatment, and determines an effect of treatment based on a change in the waveforms. As an example, the determining unit 12 extracts a detection value that can be detected from the waveform of electrocardiogram data, and determines an effect of treatment from a change of the detection value between before the treatment and after the treatment. The detection value that can be detected from the waveform of electrocardiogram data includes the P wave interval, the ST interval, and the QRS waveform itself, and an effect of treatment is determined according to changes in these detection values. At this time, a change in medical condition caused by treatment is determined as an effect of treatment, such as “medical condition improved”, “medical condition worsened”, “change in medical condition level (change in medical condition level set to a plurality of stages)”, or “no change”. For example, in a case where the person P has acute myocardial infarction, the detection value shows an increase of the ST interval, and it is determined that the medical condition has improved in accordance with the change of the ST interval. In a case where a negative Q wave is present in the QRS wave, it is determined that the medical condition has worsened.

Further, in a case where 12-lead electrocardiogram data or multiple-lead electrocardiogram data is used, the determining unit 12 counts the number of leads in the electrocardiogram data determined to be anomalous, and determines an effect of treatment based on a change of the number of leads between before the treatment and after the treatment. For example, the determining unit 12 determines an effect of treatment in the following manner: determines that the medical condition has improved in a case where the number of leads in electrocardiogram data determined to be anomalous has decreased and determines that the medical condition has worsened in a case where the number of leads has increased.

The determining unit 12 may determine an effect of treatment, not only based on a change in the waveform of electrocardiogram data and a change of the number of leads as described above, but also in consideration of record data of the person P such as basic physical data such as age, gender, height and weight, measurement data such as heart rate, body temperature and blood pressure, and medical condition data such as state of consciousness, current or past disease, a condition at the time of diagnosis and a condition at the time of examination, which are acquired from the electronic medical record. For example, the determining unit 12 may change a threshold value for determining improvement of medical condition with respect to the detection value of the waveform of electrocardiogram data mentioned above or a threshold value for determining improvement of medical condition with respect to the number of leads, or may change the type of the detection value used to determine the effect, based on the age and past medical history of the person P.

The predicting unit 13 predicts a future physical condition of the person P based on the abovementioned result of determining the effect of treatment of the person P. Specifically, the predicting unit 13 predicts the future physical condition by inputting electrocardiogram data before treatment and electrocardiogram data after treatment and the result of determining the effect of treatment into a prepared prediction model. Here, a prediction model is a model generated by learning, for each disease and for each attribute of a person, measured electrocardiogram data before and after treatment, the determined effect of treatment, and the future physical condition of the person. The physical condition of the person P may refer to the degree of recovery indicating a change in medical condition of the person P after several months, or to events that may occur to the body of the person P such as sudden death, myocardial infarction and cerebral infarction. However, the physical condition of the person P to be predicted is not limited to those mentioned above.

However, the predicting unit 13 is not necessarily limited to predicting the future physical condition of the person P using the prediction model. For example, the predicting unit 13 may predict the future condition by extracting detection values that can be detected from the waveforms of electrocardiogram data before and after treatment and comparing the detection values and the determined effect of treatment with preset reference values. As an example, the predicting unit 13 can obtain a detection value such that the anomalous waveform in electrocardiogram data before treatment decreases after treatment or gets close to the waveform in the normal state, and furthermore, the predicting unit 13 may predict, for example, that the prognosis is favorable based on the determination result that the medical condition has improved as a result of the treatment. As another example, the predicting unit 13 may predict the future physical medical condition of the person P from the determined effect of treatment. For example, in a case where the result of determining the effect of treatment indicates improvement of the medical condition, the predicting unit 13 may predict that the prognosis is favorable.

Further, the predicting unit 13 may generate a schedule for treatment for the person P based on the prediction result. For example, in a case where the prediction result indicates that an event such as sudden death, myocardial infarction or cerebral infarction will occur in the body of the person P after several months, the predicting unit 13 sets a schedule for an examination, a consultation, a surgery and the like or a medication schedule at a specified time point before several months from now. Furthermore, the predicting unit 13 may make an appointment for examination or an appointment for consultation at a corresponding medical institution according to the set schedule for examination and consultation. In this case, the predicting unit 13 is connected to a reservation system in the medical institution, and performs an appointment process to make appointments at the examination date and time and at the consultation date and time set in the schedule.

The output unit 14 transmits the content predicted as described above to the display apparatus 40 for output. In addition, as described above, in a case where the schedule for treatment is generated or the appointment process is performed, the output unit 14 may transmit information representing the content to the display apparatus 40 for output, or transmit it to an information processing apparatus of the target person P.

[Operation]

Next, the operation of the above electrocardiogram evaluation apparatus 10 will be described mainly with reference to a flowchart of FIG. 3.

First, the electrocardiogram evaluation apparatus 10 diagnoses a person P and, when predicting a future condition, acquires electrocardiogram data of the person P (step S1). At this time, the electrocardiogram evaluation apparatus 10 acquires electrocardiogram data before treatment and electrocardiogram data after treatment.

Next, the electrocardiogram evaluation apparatus 10 determines an effect of treatment from the electrocardiogram data before treatment and electrocardiogram data after treatment of the person P (step S2). For example, the electrocardiogram evaluation apparatus 10 compares the waveform of the electrocardiogram data before treatment with the waveform of the electrocardiogram data after treatment, and determines an effect of treatment based on a change in the waveform. As another example, in the case of using 12-lead electrocardiogram data or multiple-lead electrocardiogram data, the electrocardiogram evaluation apparatus 10 counts the number of leads in electrocardiogram data determined to be anomalous, and determines an effect of treatment based on a change of the number of leads between before the treatment and after the treatment.

Next, the electrocardiogram evaluation apparatus 10 predicts a future physical condition of the person P based on the result of determining the effect of the treatment for the person P (step S3). Specifically, the electrocardiogram evaluation apparatus 10 predicts the future physical condition by inputting the electrocardiogram data before and after treatment and the result of determining the effect of the treatment into a prepared prediction model. For example, the electrocardiogram evaluation apparatus 10 predicts the degree of recovery indicating a change in the medical condition of the person P and an event that may occur to the body of the person P such as sudden death, myocardial infarction or cerebral infarction after several months.

At this time, the electrocardiogram evaluation apparatus 10 may generate a schedule for treatment for the person P based on the prediction result. For example, the electrocardiogram evaluation apparatus 10 may set a schedule for the next examination, consultation and surgery, and make an appointment for examination and an appointment for consultation at a corresponding medical institution according to the set schedule for examination and consultation.

Then, the electrocardiogram evaluation apparatus 10 transmits the content predicted as described above to the display apparatus 40 for output (step S4). At this time, the electrocardiogram evaluation apparatus 10 may also transmit the generated treatment schedule to the display apparatus 40 for display, or transmit the schedule and appointment details to the information processing device of the target person P.

In this manner, the display apparatus 40 displays the predicted future physical condition of the person P who has received treatment. Such prediction result is highly accurate because it is based on the result of determining the effect of treatment from electrocardiogram data before and after treatment. Therefore, the medical professional 41 such as a doctor can take more appropriate measures by looking at the prediction results and planning subsequent treatment methods, schedules, medication, and so forth.

### Second Example Embodiment

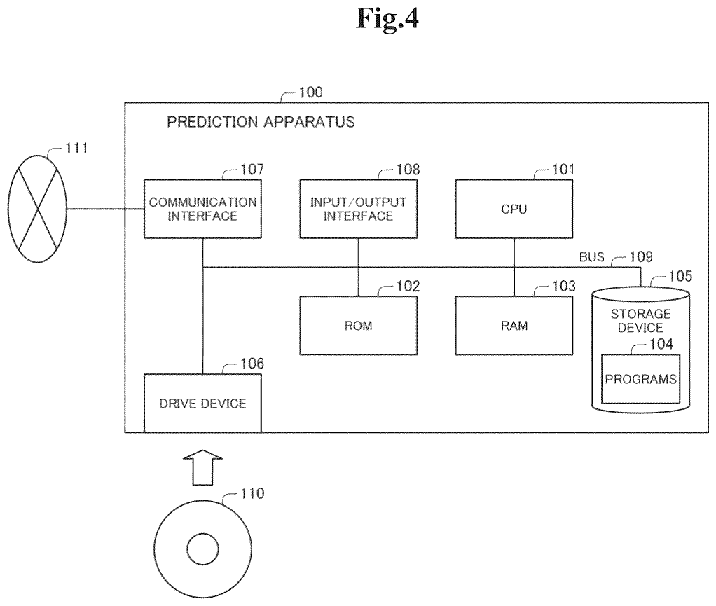

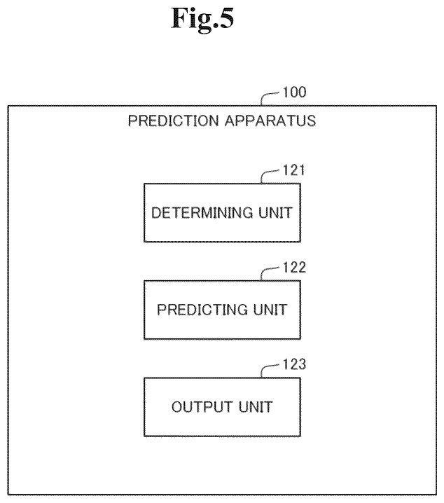

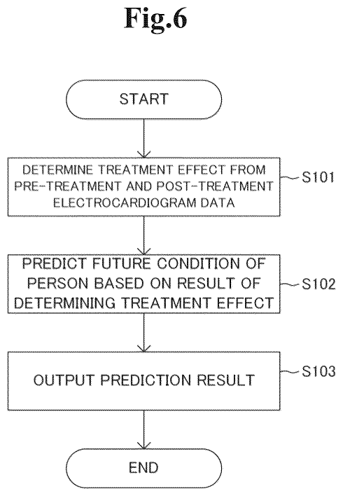

Next, a second example embodiment of the present invention will be described with reference to FIGS. 4 to 6. FIGS. 4 and 5 are block diagrams showing the configuration of a prediction apparatus in the second example embodiment, and FIG. 6 is a flowchart showing the operation of the prediction apparatus. In this example embodiment, the overview of the configurations of the electrocardiogram evaluation apparatus and the electrocardiogram evaluation method described in the above example embodiment is shown.

First, with reference to FIG. 4, the hardware configuration of a prediction apparatus 100 in this example embodiment will be described. The prediction apparatus 100 is configured with a general information processing apparatus, and as an example, has the following hardware configuration including:
a CPU (Central Processing Unit) 101 (arithmetic logic unit) (or a GPU (Graphics Processing Unit));
a ROM (Read Only Memory) 102 (memory unit);
a RAM (Random Access Memory) 103 (memory unit);
programs 104 loaded to the RAM 103;
a storage device 105 storing the programs 104;
a drive device 106 that reads from and writes into a storage medium 110 outside the information processing apparatus;
a communication interface 107 connected to a communication network 111 outside the information processing apparatus;
an input/output interface 108 that inputs and outputs data; and
a bus 109 that connects the respective components.

Then, the prediction apparatus 100 can construct and include a determining unit 121, a predicting unit 122, and an output unit 123 shown in FIG. 5 by acquisition and execution of the programs 104 by the CPU 101. The programs 104 are, for example, stored in advance in the storage device 105 or the ROM 102, and are loaded into the RAM 103 and executed by the CPU 101 as necessary. The programs 104 may be provided to the CPU 101 via the communication network 111, or the programs 104 may be stored in the storage medium 110 in advance and read out by the drive device 106 and provided to the CPU 101. However, the determining unit 121, the predicting unit 122, and the output unit 123 described above may be constructed using dedicated electronic circuits for realizing such means.

FIG. 4 shows an example of the hardware configuration of the information processing apparatus serving as the prediction apparatus 100, and the hardware configuration of the information processing apparatus is not limited to the above case. For example, the information processing apparatus may be configured with part of the above configuration, such as without the drive device 106.

Then, the prediction apparatus 100 executes a prediction method shown in the flowchart of FIG. 6 by the functions of the determining unit 121, the predicting unit 122, and the output unit 123 constructed by the program as described above.

As shown in FIG. 6, the prediction apparatus 100 executes processes to:
determine an effect of treatment from electrocardiogram data before and after the treatment for a disease acquired from a person (step S101);
predict a future physical condition of the person based on a result of determining the effect of the treatment (step S102); and
output a result of predicting (step S103).

Here, electrocardiogram data may be any electrocardiogram such as a 12-lead electrocardiogram or a monitored electrocardiogram. Then, the determining unit 121 determines an effect of treatment from, for example, a change in the waveform of the electrocardiogram data or a change of the number of anomalous leads. An effect of treatment to be determined represents a change in the medical condition, for example, whether the medical condition has improved or worsened. The predicting unit 122 then predicts a future physical condition of the person using the electrocardiogram data before and after the treatment, together with the effect of the treatment described above. For example, the predicting unit 122 predicts a future physical condition of the person by inputting the electrocardiogram data before and after the treatment and the determined treatment effect into a prepared prediction model. The condition to be predicted may be, for example, a change in the medical condition after several months or a possible event. Then, the output unit 123 outputs the prediction result.

Configured as described above, the present invention enables evaluation of electrocardiogram data using criteria suited to circumstances under which the electrocardiogram of a person has been measured, and it is possible to obtain more accurate evaluation results. As a result, for example, it is possible to obtain electrocardiogram evaluation results that can accurately distinguish between a healthy young person and a patient with acute myocardial infarction, whose electrocardiograms may have similar waveforms, or between patients with different diseases.

The abovementioned programs can be stored and provided to a computer using various types of non-transitory computer-readable mediums. Non-transitory computer-readable mediums include various types of tangible storage mediums. Examples of non-transitory computer-readable mediums include a magnetic recording medium (e.g., floppy disk, magnetic tape, hard disk drive), a magneto-optical recording medium (e.g., magneto-optical disk), a CD-ROM (Read Only Memory), a CD-R, a CD-R/W, and a semiconductor memory (e.g., mask ROM, PROM (Programmable ROM), EPROM (Erasable PROM), flash ROM, and RAM (Random Access Memory)). The program may also be provided to a computer by various types of transitory computer-readable mediums. Examples of transitory computer-readable mediums include an electrical signal, an optical signal, and an electromagnetic wave. The temporary computer-readable medium can provide the program to the computer via a wired communication path such as an electric wire or an optical fiber, or via a wireless communication path.

Although the present invention has been described above with reference to the above example embodiments, the present invention is not limited to the above example embodiments. The configuration and details of the present invention can be modified in various ways that are understandable to a person skilled in the art within the scope of the present invention. Moreover, at least one or more of the functions of the determining unit 121, the predicting unit 122 and the output unit 123 described above may be executed by an information processing apparatus installed and connected anywhere on the network, that is, may be executed by so-called cloud computing.

### Supplementary Notes

The whole or part of the example embodiments disclosed above can be described as the following supplementary notes. The overview of the configurations of a prediction method, a prediction apparatus, and a program according to the present invention will be described below. However, the present invention is not limited to the following configurations.

### Supplementary Note 1

A prediction method comprising:
determining an effect of treatment for a disease from electrocardiogram data before the treatment and electrocardiogram data after the treatment acquired from a person;
predicting a future condition of a body of the person based on a result of determining the effect of the treatment; and
outputting a result of predicting.

### Supplementary Note 2

The prediction method according to Supplementary Note 1, comprising
determining the effect of the treatment based on a change in waveform in the electrocardiogram data.

### Supplementary Note 3

The prediction method according to Supplementary Note 1 or 2, comprising determining the effect of the treatment based on a change of number of leads determined to be anomalous in the electrocardiogram data.

### Supplementary Note 4

The prediction method according to any one of Supplementary Notes 1 to 3, comprising determining the effect of the treatment based on record data on the body of the person.

### Supplementary Note 5

The prediction method according to any one of Supplementary Notes 1 to 4, comprising predicting a change in the disease of the person.

### Supplementary Note 6

The prediction method according to any one of Supplementary Notes 1 to 5, comprising predicting an event that may occur to the person.

### Supplementary Note 7

The prediction method according to any one of Supplementary Notes 1 to 6, comprising predicting the future condition of the body of the person based on the electrocardiogram data before and after the treatment for the disease acquired from the person and on the result of determining the effect of the treatment.

### Supplementary Note 7.1

The prediction method according to any one of Supplementary Notes 1 to 7, comprising predicting the future condition of the body of the person based on:
a prediction model generated by learning a relation of electrocardiogram data before and after treatment for a disease measured from a predetermined person, an effect of the treatment of the predetermined person determined based on the electrocardiogram data, and a condition of a body after the treatment of the predetermined person;
electrocardiogram data before and after the treatment for the disease newly acquired from the person; and
a result of determining an effect of the treatment based on the electrocardiogram data newly acquired from the person.

### Supplementary Note 7.2

The prediction method according to Supplementary Note 7.1, wherein the prediction model is a model generated for each disease of the predetermined person or for each attribute of the predetermined person.

### Supplementary Note 8

The prediction method according to any one of Supplementary Notes 1 to 7, comprising generating a schedule for the treatment of the person based on the result of predicting.

### Supplementary Note 9

The prediction method according to any one of Supplementary Notes 1 to 8, comprising performing an appointment process for the treatment of the person based on the result of predicting.

### Supplementary Note 10

A prediction apparatus comprising:
a determining unit that determines an effect of treatment for a disease from electrocardiogram data before the treatment and electrocardiogram data after the treatment acquired from a person;
a predicting unit that predicts a future condition of a body of the person based on a result of determining the effect of the treatment; and
an output unit that outputs a result of predicting.

### Supplementary Note 11

The prediction apparatus according to Supplementary Note 10, wherein the determining unit determines the effect of the treatment based on a change in waveform in the electrocardiogram data.

### Supplementary Note 12

The prediction apparatus according to Supplementary Note 10 or 11, wherein the determining unit determines the effect of the treatment based on a change of number of leads determined to be anomalous in the electrocardiogram data.

### Supplementary Note 13

The prediction apparatus according to any one of Supplementary Notes 10 to 12, wherein the determining unit determines the effect of the treatment based on record data on the body of the person.

### Supplementary Note 14

The prediction apparatus according to any one of Supplementary Notes 10 to 13, wherein the predicting unit predicts a change in the disease of the person.

### Supplementary Note 15

The prediction apparatus according to any one of Supplementary Notes 10 to 14, wherein the predicting unit predicts an event that may occur to the person.

### Supplementary Note 16

The prediction apparatus according to any one of Supplementary Notes 10 to 15, wherein the predicting unit predicts the future condition of the body of the person based on the electrocardiogram data before and after the treatment for the disease acquired from the person and on the result of determining the effect of the treatment.

### Supplementary Note 16.1

The prediction apparatus according to any one of Supplementary Notes 10 to 16, wherein the predicting unit predicts the future condition of the body of the person based on:
a prediction model generated by learning a relation of electrocardiogram data before and after treatment for a disease measured from a predetermined person, an effect of the treatment of the predetermined person determined based on the electrocardiogram data, and a condition of a body after the treatment of the predetermined person;
electrocardiogram data before and after the treatment for the disease newly acquired from the person; and
a result of determining an effect of the treatment based on the electrocardiogram data newly acquired from the person.

### Supplementary Note 16.2

The prediction apparatus according to Supplementary Note 16.1, wherein the prediction model is a model generated for each disease of the predetermined person or for each attribute of the predetermined person.

### Supplementary Note 17

The prediction apparatus according to any one of Supplementary Notes 10 to 16, wherein the predicting unit generates a schedule for the treatment of the person based on the result of predicting.

### Supplementary Note 18

The prediction apparatus according to any one of Supplementary Notes 10 to 17, wherein the predicting unit performs an appointment process for the treatment of the person based on the result of predicting.

### Supplementary Note 19

A non-transitory computer-readable storage medium storing a program, the program comprising instructions for causing a computer to execute processes to:
determine an effect of treatment for a disease from electrocardiogram data before the treatment and electrocardiogram data after the treatment acquired from a person;
predict a future condition of a body of the person based on a result of determining the effect of the treatment; and
output a result of predicting.

### REFERENCE SIGNS LIST

10 electrocardiogram evaluation apparatus
11 electrocardiogram acquiring unit
12 determining unit
13 predicting unit
14 output unit
16 data storing unit
20 electronic medical record apparatus
30 electrocardiogram measurement apparatus
40 display apparatus
P person
100 prediction apparatus
101 CPU
102 ROM
103 RAM
104 programs
105 storage device
106 drive device
107 communication interface
108 input/output interface
109 bus
110 storage medium
111 communication network
121 determining unit
122 predicting unit
123 output unit

What is claimed is:

1. A prediction method comprising:
determining an effect of treatment for a disease from electrocardiogram data before the treatment and electrocardiogram data after the treatment acquired from a person;
when predicting a future condition of a body of the person based on a result of determining the effect of the treatment, predicting the future condition of the body of the person based on: a prediction model generated through machine learning of a relation of electrocardiogram data before and after treatment for a disease measured from a predetermined person, an effect of the treatment of the predetermined person determined based on the electrocardiogram data, and a condition of a body after the treatment of the predetermined person; electrocardiogram data before and after the treatment for the disease newly acquired from the person; and a result of determining an effect of the treatment based on the electrocardiogram data newly acquired from the person; and
outputting a result of predicting.

2. The prediction method according to claim 1, comprising
determining the effect of the treatment based on a change in waveform in the electrocardiogram data.

3. The prediction method according to claim 1, comprising
determining the effect of the treatment based on a change of number of leads determined to be anomalous in the electrocardiogram data.

4. The prediction method according to claim 1, comprising
determining the effect of the treatment based on record data on the body of the person.

5. The prediction method according to claim 1, comprising
predicting a change in the disease of the person.

6. The prediction method according to claim 1, comprising
predicting an event that may occur to the person.

7. The prediction method according to claim 1, comprising
predicting the future condition of the body of the person based on the electrocardiogram data before and after the treatment for the disease acquired from the person and on the result of determining the effect of the treatment.

8. (canceled)

9. The prediction method according to claim 1, wherein
the prediction model is a model generated for each disease of the predetermined person or for each attribute of the predetermined person.

10. The prediction method according to claim 1, comprising
generating a schedule for the treatment of the person based on the result of predicting.

11. The prediction method according to claim 1, comprising
performing an appointment process for the treatment of the person based on the result of predicting.

12. A prediction apparatus comprising:
at least one memory storing processing instructions; and
at least one processor configured to execute the processing instructions to:
determine an effect of treatment for a disease from electrocardiogram data before the treatment and electrocardiogram data after the treatment acquired from a person;
when predicting a future condition of a body of the person based on a result of determining the effect of the treatment, predicting the future condition of the body of the person based on: a prediction model generated through machine learning of a relation of electrocardiogram data before and after treatment for a disease measured from a predetermined person, an effect of the treatment of the predetermined person determined based on the electrocardiogram data, and a condition of a body after the treatment of the predetermined person; electrocardiogram data before and after the treatment for the disease newly acquired from the person; and a result of determining an effect of the treatment based on the electrocardiogram data newly acquired from the person; and
output a result of predicting.

13-22. (canceled)

23. A non-transitory computer-readable storage medium storing a program, the program comprising instructions for causing a computer to execute processes to:
determine an effect of treatment for a disease from electrocardiogram data before the treatment and electrocardiogram data after the treatment acquired from a person;
when predicting a future condition of a body of the person based on a result of determining the effect of the treatment, predict the future condition of the body of the person based on: a prediction model generated through machine learning of a relation of electrocardiogram data before and after treatment for a disease measured from a predetermined person, an effect of the treatment of the predetermined person determined based on the electrocardiogram data, and a condition of a body after the treatment of the predetermined person; electrocardiogram data before and after the treatment for the disease newly acquired from the person; and a result of determining an effect of the treatment based on the electrocardiogram data newly acquired from the person; and
output a result of predicting.
